(19) United States
(12) Patent Application Publication
(10) Pub. No.: US 2013/0158409 A1
(43) Pub. Date: Jun. 20, 2013

(54) METHOD AND APPARATUS FOR PERFORMING ANALOG BEAMFORMING

(71) Applicants: SAMSUNG ELECTRONICS CO., LTD., Suwon-si (KR); Industry-University Cooperation Foundation Sogang University, Seoul (KR)

(72) Inventors: Bae-hyung KIM, Yongin-si (KR); In-pyo KYUNG, Seoul (KR); Jin-ho CHANG, Seoul (KR); Dong-wook KIM, Seoul (KR); Jong-keun SONG, Yongin-si (KR); Tai-kyong SONG, Seoul (KR); Yang-mo YOO, Seoul (KR); Kyung-il CHO, Seoul (KR); Seung-heun LEE, Seoul (KR)

(73) Assignees: Industry-University Cooperation Foundation Sogang University, Seoul (KR); SAMSUNG ELECTRONICS CO., LTD., Suwon-si (KR)

(21) Appl. No.: 13/716,632

(22) Filed: Dec. 17, 2012

(30) Foreign Application Priority Data
Dec. 15, 2011 (KR) 10-2011-0135770

(51) Int. Cl. A61B 8/00

(52) U.S. Cl. CPC A61B 8/4488; A61B 8/54
USPC 600/459

(57) ABSTRACT

A method and apparatus for performing analog beamforming is provided. According to the method and apparatus, the analog beamforming is effectively performed by using as small a number of devices as possible and by processing a relatively small amount of data.

### CROSS-REFERENCE TO RELATED APPLICATIONS

This application claims priority from Korean Patent Application No. 10-2011-0135770, filed on Dec. 15, 2011, in the Korean Intellectual Property Office, the disclosure of which is incorporated herein in its entirety by reference.

### BACKGROUND

1. Field

Exemplary embodiments relate to an ultrasonic image field, and more particularly, to a method and apparatus for performing analog beamforming.

2. Description of the Related Art

In general, a probe of an ultrasonic diagnostic device is manufactured as a transducer. When an ultrasonic wave which has a frequency which falls within a range of between several kHz and several hundreds of MHz is transmitted from a probe of a three-dimensional (3D) image detecting apparatus into a predetermined portion of the human body of a patient, the ultrasonic wave is partially reflected from materials between various different tissues. In particular, ultrasonic waves are reflected from an internal portion of the human body in which a density is changed, for example, blood cells in blood plasma, small structures in organs, and other such internal structures. The reflected ultrasonic waves cause an oscillation in a transducer of a probe, and the transducer outputs electrical pulses based on the oscillation. The electrical pulses are converted into an image. However, because an intensity of a signal of the reflected ultrasonic waves is very weak and a signal to noise ratio (SNR) of the signal is low, technologies for increasing the intensity and SNR of the signal of the ultrasonic wave are required in order to convert the signal into image information. One of these technologies is beamforming.

Beamforming entails increasing an intensity of a signal by super-positioning signals when the signals are transmitted and received via a plurality of transducer elements. FIG. 1 is a conceptual diagram which illustrates a transmission beamforming operation which is performed by using a one-dimensional (1D) transducer array. A single point from which image information is to be obtained is referred to as a focal point. Because a plurality of transducer elements are arranged in a straight line, there is a difference in a respective distance between each respective one of the transducer elements and the focal point. Thus, because periods of time when signals are transmitted and returned in respective transducer elements are different, when the received signals are superposed on each other, phases of the received signals do not correspond to each other, and thus, the signals may not be amplified. As a result, in order to perform beamforming, there is a need for a process of adjusting phases of signals whose phases do not correspond to each other.

A method for adjusting phases is performed by delaying transmitted and received signals. A beamforming method is classified based on a corresponding method for delaying a signal. The beamforming method is classified as one of an analog beamforming method and a digital beamforming method. In the analog beamforming method, a signal is delayed by using a circuit device. Conversely, in the digital beamforming method, a signal is delayed by digitizing and then storing the signal, and then reading data after a predetermined time elapses.

### SUMMARY

Provided are methods and apparatuses for performing analog beamforming for reducing a required number of hardware devices and reducing a required amount of calculation which is performed by a system and a correspondingly required amount of memory. In addition, provided are computer readable recording media having recorded thereon respective programs for executing the methods.

Additional aspects will be set forth in part in the description which follows and, in part, will be apparent from the description, or may be learned by practice of the presented exemplary embodiments.

According to an aspect of an exemplary embodiment, an apparatus for performing analog beamforming by using ultrasonic signals that are received by a transducer array having a plurality of transducer elements includes a delay controller which outputs respective first delay controlling information which indicates a number of delays of a first time interval and respective second delay controlling information which indicates a number of delays of a second time interval, with respect to each respective one of the ultrasonic signals and a respective predetermined position of a scanning region; first analog devices, each respective one of which selectively delays a corresponding one of the received ultrasonic signals by a respective first amount of time which is less than or equal to the first time interval at least once, based on the corresponding first delay controlling information; second analog devices, each respective one of which selectively delays a corresponding one of the selectively delayed ultrasonic signals by a respective second amount of time which is less than or equal to the second time interval at least once, based on the corresponding second delay controlling information; and an adder which sums the ultrasonic signals which have been selectively delayed by the second analog devices.

According to another aspect of an exemplary embodiment, a method for performing analog beamforming by using ultrasonic signals that are received by a transducer array having a plurality of transducer elements includes obtaining respective first delay controlling information which indicates a number of delays of a first time interval and respective second delay controlling information which indicates a number of delays of a second time interval, with respect to each respective one of the ultrasonic signals and a respective predetermined position of a scanning region; selectively delaying or transmitting each respective one of the received ultrasonic signals by a respective first amount of time which is less than or equal to the first time interval at least once, based on the corresponding first delay controlling information; selectively delaying or transmitting each respective one of the delayed or transmitted ultrasonic signals by a respective second amount of time which is less than or equal to the second time interval at least once, based on the corresponding second delay controlling information; and summing the ultrasonic signals that have twice been selectively delayed or transmitted.

According to another aspect of an exemplary embodiment, a non-transitory computer readable recording medium has recorded thereon a program for executing the method.

### BRIEF DESCRIPTION OF THE DRAWINGS

These and/or other aspects will become apparent and more readily appreciated from the following description of the exemplary embodiments, taken in conjunction with the accompanying drawings of which:

FIG. 1 is a conceptual diagram which illustrates a transmission beamforming operation which is performed by using a one-dimensional (1D) transducer array;

FIG. 2 is a diagram which illustrates a two-dimensional (2D) transducer array transmitter and receiver in a probe, according to an exemplary embodiment;

FIG. 3 is a structural diagram which illustrates an ultrasonic image system, according to an exemplary embodiment;

FIG. 4 illustrates a case in which a position of a focal point is changed, according to an exemplary embodiment;

FIG. 5 is a structural diagram which illustrates a probe and a beamforming apparatus shown in FIG. 3, according to an exemplary embodiment;

FIG. 6 is a schematic perspective view of a charge-coupled device (CCD), according to an exemplary embodiment;

FIGS. 7A, 7B, and 7C are diagrams which illustrate a relationship between an input clock signal and a time delay resolution in a CCD, according to an exemplary embodiment;

FIG. 8 illustrates a case in which an error may arise in a time delay when a time delay resolution is low, according to an exemplary embodiment;

FIG. 9 is a structural diagram which illustrates a clock generator, according to an exemplary embodiment;

FIGS. 10A and 10B are diagrams which illustrate a single channel included in a received signal delayer for correcting a time delay error, according to an exemplary embodiment;

FIG. 11 is diagram which illustrates a method for correcting a time delay error by using a received signal delayer as shown in FIGS. 10A and 10B, according to an exemplary embodiment;

FIG. 12 is diagram which illustrates a case in which a received signal delayer as shown in FIGS. 10A and 10B performs a time delay via two processes, according to an exemplary embodiment;

FIGS. 13A and 13B are diagrams which illustrate an internal structure of an output selection controller as shown in FIGS. 10A and 10B, according to exemplary embodiments;

FIG. 14 is a diagram which illustrates an internal structure of an output selection controller as shown in FIGS. 10A and 10B, according to another exemplary embodiment;

FIG. 15 is a diagram which illustrates an internal structure of a tri-state buffer block as shown in FIG. 14, according to an exemplary embodiment; and

FIG. 16 is diagram which illustrates a method for obtaining a planar image via a 2D transducer array, according to an exemplary embodiment.

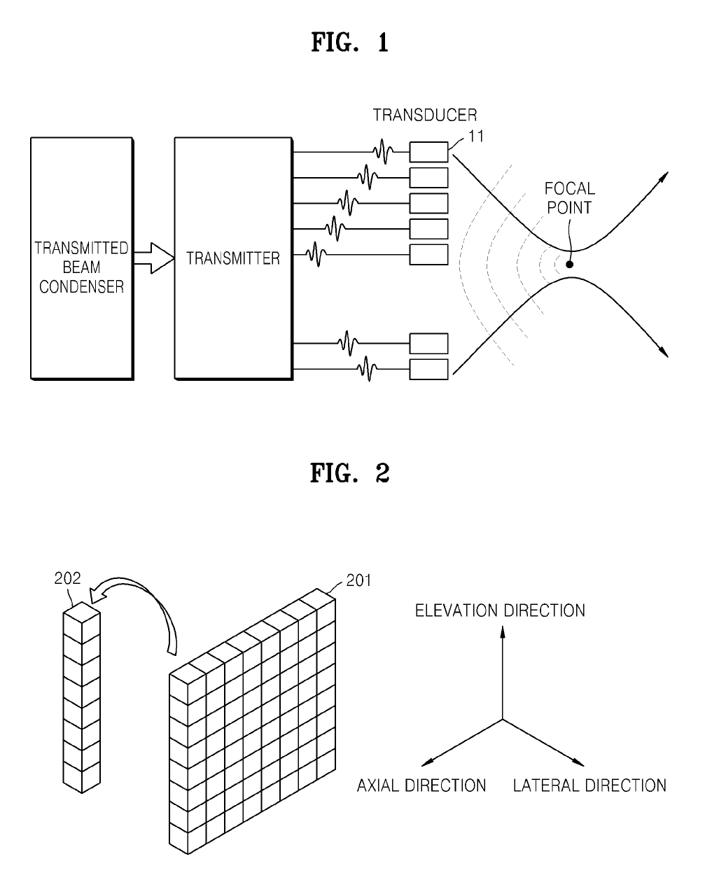

### DETAILED DESCRIPTION

Reference will now be made in detail to exemplary embodiments, examples of which are illustrated in the accompanying drawings, wherein like reference numerals refer to like elements throughout. In this regard, the exemplary embodiments may have different forms and should not be construed as being limited to the descriptions set forth herein. Accordingly, the exemplary embodiments are merely described below, by referring to the figures, to explain aspects of the present disclosure.

FIG. 2 is a diagram which illustrates a two-dimensional (2D) transducer array transmitter and receiver in a probe, according to an exemplary embodiment. A reference numeral 201 indicates an arrangement of a 2D transducer array. A reference numeral 202 indicates a single 2D transducer array which has a first elevation direction.

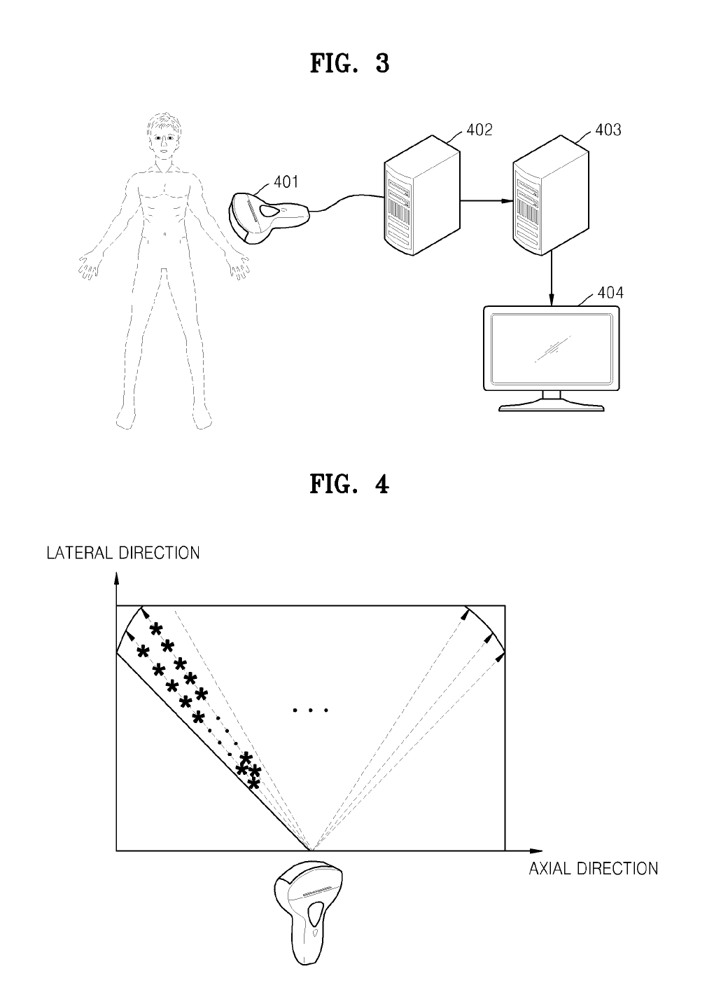

FIG. 3 is a structural diagram which illustrates an ultrasonic image system according to an exemplary embodiment. Referring to FIG. 3, the ultrasonic image system shown in FIG. 3 includes a probe 401, a beamforming apparatus 402, an image generating apparatus 403, and an image displaying apparatus 404. The probe 401 includes a transducer array. When the probe 401 receives electrical signals, which respectively correspond to transducer elements included in the transducer array, from the beamforming apparatus 402, the probe 401 converts the electrical signals into source signals by using the transducer elements. In this case, the probe 401 uses a plurality of transducer elements rather than a single transducer element, because the source signals generated by the probe 401 are too weak to be used as image information. Hereinafter, each respective single data line which is connected to each respective transducer device will be referred to as a channel. Thus, the number of channels formed corresponds to the number of transducer elements. Thus, superposition of the source signals is used to obtain sufficient signal intensity. For example, in an exemplary embodiment, a one-dimensional (1D) transducer array includes 128 transducer elements and the probe 401 converts 128 electrical signals into 128 source signals by using respective transducer elements. Hereinafter, it is assumed that a 1D transducer array 202 device includes 128 transducer elements.

As shown in FIG. 3, the beamforming apparatus 402 may be configured to be separated from the probe 401 so as to transmit and receive data to and from the probe 401, or alternatively, the beamforming apparatus 402 may be configured to be integrated with the probe 401. The beamforming apparatus 402 outputs 128 electrical signals to be converted into source signals by transducer elements of the probe 401. In this case, the 128 source signals generated by the probe 401 must simultaneously reach a focal point so as to be superposed on each other at the focal point. In a case of a 1D transducer array, transducer elements of the probe 401 are arranged in a straight line at an end portion of the probe 401. Thus, there is a distance difference between respective distances from a predetermined focal point to each respective transducer device, and a time difference between respective times required for a source signal to reach the focal point from each respective transducer device. As a result, in order for the 128 source signals generated by the probe 401 to simultaneously reach the focal point, a transducer that is relatively far from the focal point may generate a source signal at a relatively early time, and a transducer that is relatively close to the focal point may generate a source signal at a later time. Thus, the beamforming apparatus 402 outputs the respective electrical signals at different respective times, such that each of the electrical signals is converted into a corresponding source signal at a different respective time by the respective transducer elements. As described above, the probe 401 outputs the 128 electrical signals as 128 source signals.

When the source signals reach the focal point, the source signals may be reflected from tissue of the human body at the focal point or a peripheral portion thereof. When the reflected signals reach a transducer array, the respective transducer elements of the probe 401 convert the reflected signals into electrical signals. Thus, the probe 401 outputs the electrical signals, the number of which corresponds to the number of the transducer elements.

When the probe 401 generates 128 electrical signals converted from the reflected signals, the beamforming apparatus 402 receives the 128 electrical signals, adjusts respective phases of each of the 128 electrical signals, and superposes the 128 electrical signals upon each other in order to generate a single signal. In this case, it is required that the beamforming apparatus 402 adjust the respective phases of each of the 128 electrical signals such that the 128 electrical signals are superposed on each other, because respective time points when the reflected signals reach the 128 transducer elements are different. The beamforming apparatus 402 converts the single signal which is generated by superposing the signals upon each other into a digital signal by using an analog-digital (AD) converter. The beamforming apparatus 402 stores an intensity of the digital signal as image information relating to a single focal point. The beamforming apparatus 402 outputs respective intensity information relating to the digital signal for each focal point to the image generating apparatus 403 by using the above-described method while changing positions of focal points.

FIG. 4 illustrates a case in which a position of a focal point is changed, according to an exemplary embodiment. FIG. 4 shows an example in which an ultrasonic image is generated with respect to a depth which is measured in an axial direction of between several centimeters and several tens of centimeters (e.g., 20 cm) based on a position of a transducer array of the probe 401 and a width which is measured in a lateral direction of between several centimeters and several tens of centimeters (e.g., 10 cm) based on a central portion of the transducer array For example, the size of the ultrasonic image may be equal to 10 cm (lateral direction) X 20 cm. An arrow indicated by a dotted line denotes a scan line, and an asterisk (*) denotes a focal point. If a scan line angle based on the probe 401 is determined as being equal to 45 degrees on the left side and 45 degrees on the right side in the lateral direction, a starting point of the scan line may be fixed to the central portion of the probe 401, and an orientation of the scan line may be changed by as much as a predetermined angle to determine the scan line. When the scan line is determined, positions of condensing lines may be changed such that the condensing lines may be arranged at predetermined spatial intervals, for example, an interval of 1 millimeter. Likewise, image information relating to an entire image may be generated while a position of a scan focal point is changed with respect to the entire image.

When the image generating apparatus 403 receives coordinate information relating to each focal point and an intensity of a digital signal which corresponds to the coordinate information from the beamforming apparatus 402, the image generating apparatus 403 determines an intensity of a signal based on the received coordinate information and the corresponding intensity of the digital signal, converts the intensity of the signal into brightness information, and outputs the brightness information to the image displaying apparatus 404. The image displaying apparatus 404 displays an image on a screen by using the brightness information to generate an image pixel, which is output from the image generating apparatus 403.

Likewise, the beamforming apparatus 402 delays an output time of an electrical signal in order to vary points of time when 128 electrical signals are output such that source signals may be superposed on each other with respect to a predetermined focal point. In addition, when the probe 401 converts a reflective signal which is reflected from a focal point into an electrical signal, the probe 401 then adjusts a phase of the electrical signal. Phases of signals are adjusted such that the signals may be superposed on each other, which phase adjustment process is referred to as beamforming.

In order to perform beamforming, the beamforming apparatus 402 delays an output time of an electrical signal. Hereinafter, a maximum amount of time by which an output time of an electrical signal may be delayed is referred to as a maximum delay time, and a time delay resolution indicates a minimum iteration of time by which an output time of an electrical signal may be delayed. For example, if a time delay resolution is equal to 1 Hz, because the beamforming apparatus 402 may delay an output time of an electrical signal on a one second-by-one second basis, the beamforming apparatus 402 may delay an output time of an electrical signal by an amount of time which is equal to any integral multiple of one second, i.e., one second, two seconds, three seconds, and so on. However, the beamforming apparatus 402 is not capable of delaying an output time of an electrical signal by an amount of time which is not an integral multiple of the minimum iteration of time, such as, for example, 1.5 seconds. As another example, if a time delay resolution is equal to 10 Hz, because the beamforming apparatus 402 may delay an output time of an electrical signal on a 0.1 second-by-0.1 second basis, the beamforming apparatus 402 may delay an output time of an electrical signal by an amount of time which is equal to any integral multiple of 0.1 seconds, i.e., 0.1 seconds, 0.2 seconds, 0.3 seconds, and so on. As a time delay resolution is reduced (i.e., the value of the time delay resolution, as expressed in Hertz, is increased), the number of focal points on which an image is focused is increased, and lateral direction resolution may be further improved. The lateral direction is illustrated in FIG. 2.

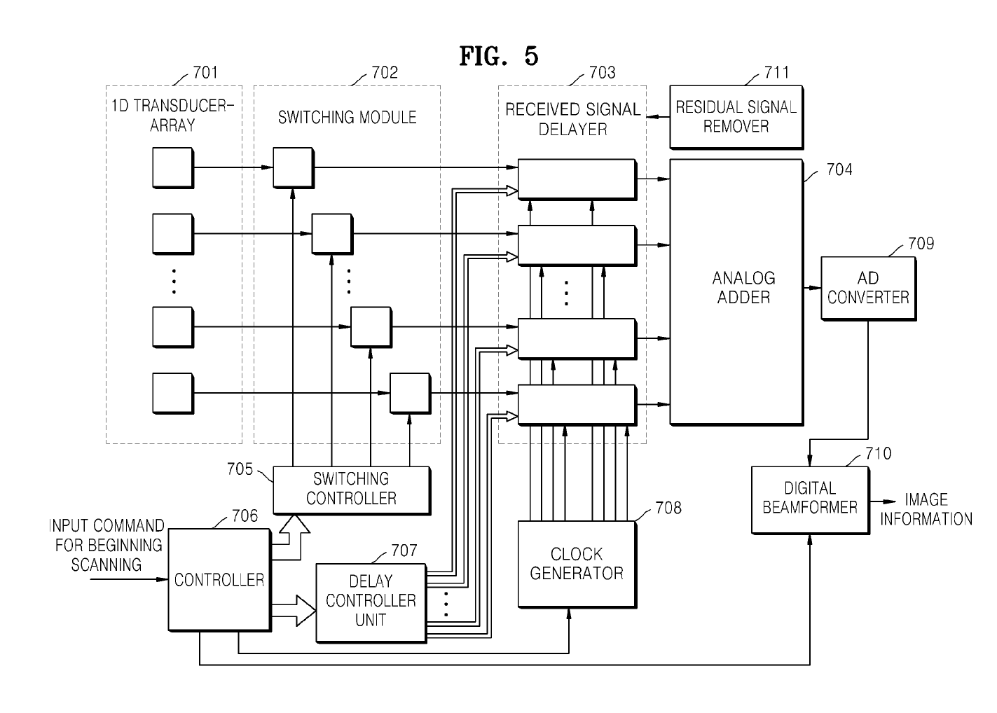

FIG. 5 is a structural diagram which illustrates the probe 401 and the beamforming apparatus 402 as shown in FIG. 3, according to an exemplary embodiment. As described above, the probe 401 and the beamforming apparatus 402 may be integrated with each other. The probe 401 and the beamforming apparatus 402 include a 1D transducer array 701, a switch module 702, a received signal delayer 703, an analog adder 704, a switching controller 705, a controller 706, a delay controller 707, a clock generator 708, an AD converter 709, a digital beamformer 710, and a residual signal remover 711.

The 2D transducer array 202, which is shown in FIG. 2, is disposed in the probe 401. In addition, the 1D transducer array 701 shown in FIG. 5 is a single transducer array which has an elevation direction with respect to the 2D transducer array 202. The 1D transducer array 701 includes a piezoelectric transducer which transmits and receives a signal. When the 1D transducer array 701 receives a signal, the 1D transducer array 701 detects a source signal which is reflected from a focal point, converts the reflected signal into an electrical pulse, and outputs the electrical pulse to the received signal delayer 703.

The switch module 702 does not output a signal which is received from the 1D transducer array 701 until the switch module 702 receives a signal for opening a switch from the switching controller 705. When the switch module 702 receives the signal for opening a switch from the switching controller 705, the switch module 702 outputs the signal received from the 1D transducer array 701 to the received signal delayer 703.

The received signal delayer 703 includes a respective element which is provided for each respective channel of the 1D transducer array 701, and delays a signal that is received from the switch module 702 by an amount of time which is determined from delay information that is input for each respective channel by the delay controller 707.

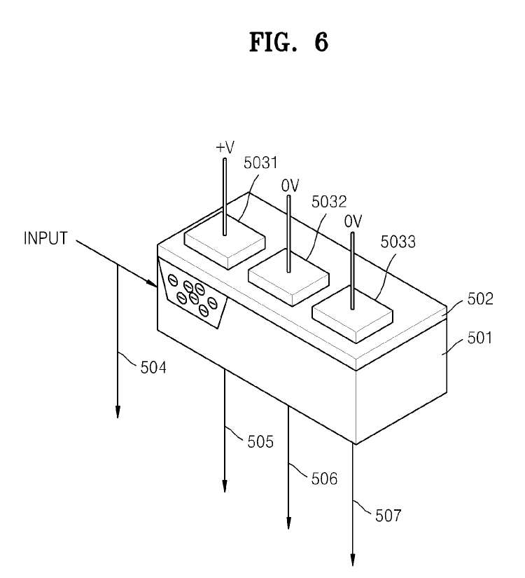

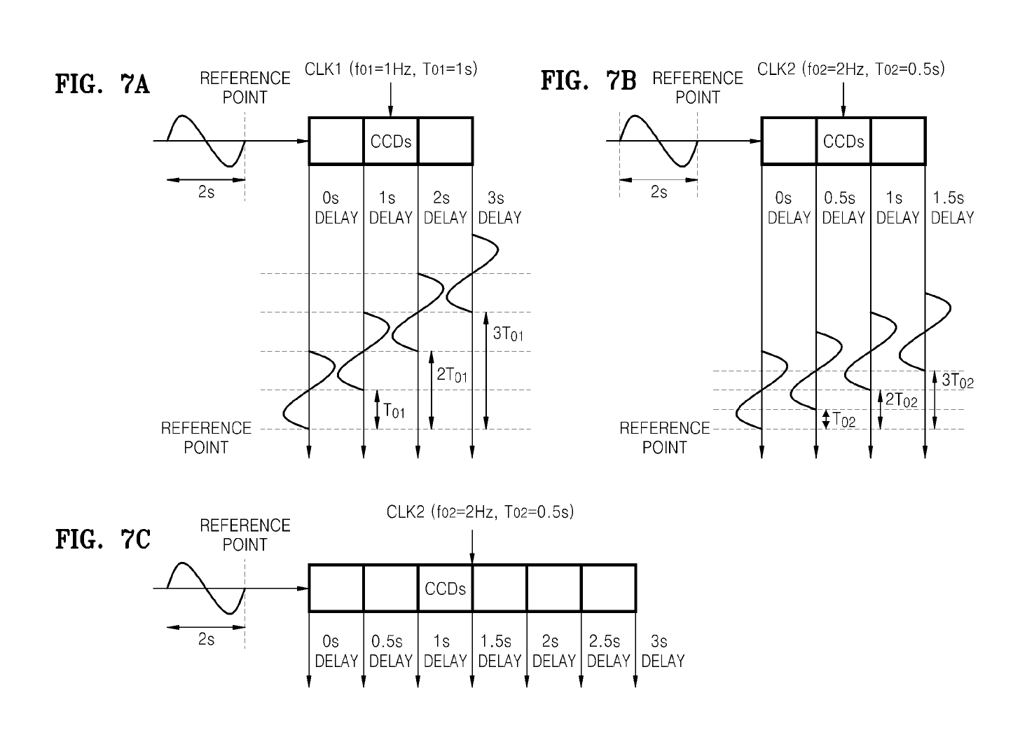

The received signal delayer 703 may adjust time delay resolution and may include, for example, a charge-coupled device (CCD). FIG. 6 is a schematic perspective view of a CCD, according to an exemplary embodiment. The CCD is a semiconductor integrated circuit which uses accumulation and movement of charges, and is configured in such a way that an insulating layer 502 having a thickness of about 0.1 mm is formed on a semiconductor surface 501, and metal electrodes 5031, 5032, and 5033 are disposed on the insulating layer 502. The CCD controls voltages of the metal electrodes 5031, 5032, and 5033 in order to sequentially transmit accumulating charges. The insulating layer 502 may be formed, for example, of SiO₂. When a signal is input to the CCD, the CCD outputs a signal 504 without any delay. A voltage may be repeatedly applied to and/or not applied to a metal electrode. When a voltage is applied to the metal electrode, electric charges accumulate below a portion of the insulating layer 502 which is positioned directly below the metal electrode to which the voltage is applied. As shown in FIG. 6, when a voltage is applied to the metal electrode 5031, input charges accumulate below a portion of the insulating layer 502 which is positioned directly below the metal electrode 5031. When electric charges accumulate on the metal electrode 5031 and the voltage which had previously been applied to the metal electrode 5031 is no longer applied, if a voltage is simultaneously applied to the metal electrode 5032, the electric charges are shifted to a portion of the insulating layer 502 which is disposed directly below the metal electrode 5032. According to the same principle as described above, when the voltage which had previously been applied to the metal electrode 5032 is no longer applied and a voltage is applied to the metal electrode 5033, electric charges are shifted to a portion of the insulating layer 502 which is positioned directly below the metal electrode 5033. Accordingly, when voltage application is controlled such that voltages are applied with a predetermined time difference therebetween, outputs 505, 506, and 507 having correspondingly delayed output times may be obtained. The above-described operation of the CCD shown in FIG. 6 may be expressed in diagram form, for example, as shown in FIG. 7A. Referring to FIG. 7A, the number of boxes corresponds to the number of electrodes. By using three outputs which correspond to electrodes and an additional output which has no delay, similar as an input, a total of four outputs may be obtained. In FIG. 7A, the four outputs which are illustrated correspond to the outputs 504, 505, 506, and 507, as shown in FIG. 6.

FIGS. 7A, 7B, and 7C are diagrams which illustrate a relationship between an input clock signal and a time delay resolution in a CCD, according to an exemplary embodiment. In this case, an input clock signal is used as a voltage for controlling metal electrodes arranged on a CCD. FIG. 7A shows time delays of outputs of the CCD when an input clock signal CLK1, which has a frequency f01 which is equal to 1 Hz and a period T01 which is equal to 1s, is applied to the CCD. A first output of the CCD is obtained with a zero time delay, a second output is obtained with a time delay which is equal to T01, a third output is obtained with a time delay which is equal to 2T01, and a fourth output is obtained with a time delay which is equal to 3T01. FIG. 7B shows time delays of outputs of the CCD when an input clock signal CLK2, which has a frequency f02 which is equal to 2 Hz and a period T02 which is equal to 0.5 s, is applied to the CCD. In this case, time delays of outputs are integral multiples of T02. Thus, when the CCD is used, the received signal delayer 703 may adjust the time delay resolution by adjusting a clock signal which is input to the received signal delayer 703. In particular, as a frequency of an input clock signal is doubled, the time delay resolution is also doubled correspondingly.

With regard to a maximum delay time, although the same CCD is used, the received signal delayer 703 delays an output by a maximum delay time of three seconds in the example illustrated in FIG. 7A, but delays an output by a maximum delay time of 1.5 seconds in the example illustrated in FIG. 7B. As a result, in order for the received signal delayer 703 to increase a time delay resolution and to ensure the same maximum delay time, the number of CCDs that are connected in series to each other must be increased, as illustrated in FIG. 7C. In the example illustrated in FIG. 7C, the received signal delayer 703 obtains the same maximum delay time as in the example illustrated in FIG. 7A, while also using the same time delay resolution as in the example illustrated in FIG. 7B. By reducing the time delay resolution, the number of CCDs that are connected in series to each other may be reduced. In an exemplary embodiment, the number of CCDs that are connected in series to each other is reduced while the same maximum delay time is obtained.

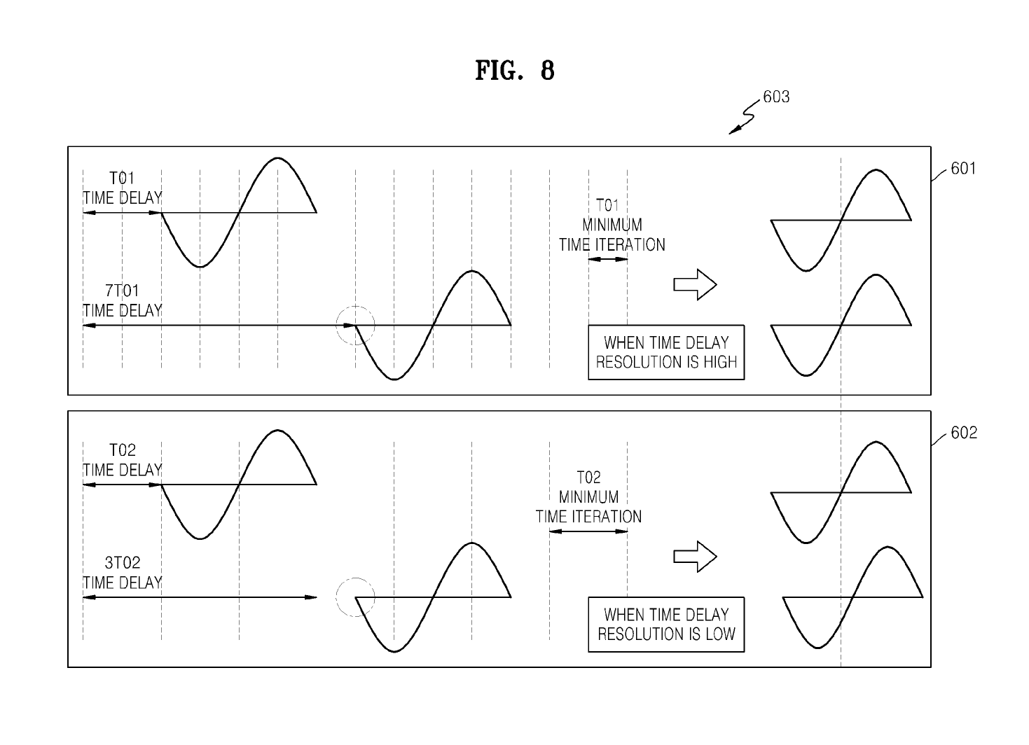

FIG. 8 illustrates a case in which an error may arise in a time delay when a time delay resolution is low, according to an exemplary embodiment. In an exemplary embodiment, the number of CCDs that are connected in series to each other is reduced but a quality level of beamforming performance is maintained. A reference numeral 601 indicates a result of a case in which a time delay of two channels is performed in a signal when a minimum time iteration of the received signal delayer 703 is equal to T01. A reference numeral 602 indicates a result of a case in which a time delay of two channels is performed in the same signal as the signal as used the result 601 when a minimum time iteration is equal to T02, which is equal to 2T01. Thus, the time delay resolution in the result 601 is equal to double the time delay resolution in the result 602. With regard to the result 601, a time delay is performed by the received signal delayer 703 such that centers of two signals accurately correspond to each other. However, with regard to the result 602, in which a time delay resolution with respect to the same signal is low and a time delay is performed by using a relatively high minimum time iteration of T02, the centers of the two signals do not correspond to each other. Thus, when the received signal delayer 703 performs a time delay with a low time delay resolution, an error may arise in a time delay, and thus, a corresponding image quality may be reduced.

The analog adder 704 superposes electrical signals on each other that are delayed by the received signal delayer 703 in order to generate a single signal. The generated signal is output to the AD converter 709.

Based on a control executed by the controller 706, the switching controller 705 receives coordinate information relating to a focal point, calculates a respective distance between the focal point and each respective channel, and outputs a signal, which is used for turning on a switch, to the switch module 702 only at a time point when the signal is reflected and reaches each respective channel.

When the controller 706 receives a command for beginning scanning from a user or from another device, the controller 706 outputs coordinate information relating to a focal point to the switching controller 705, the delay controller 707, the clock generator 708, and the residual signal remover 711 while sequentially changing the focal point. In this case, the controller 706 may receive a command to begin scanning via a switch included in the probe 401 as an input from a user. In addition, the controller 706 may receive a command to begin scanning from another device when the probe 401 comes in contact with the human body via a contact sensor included in the probe 401.

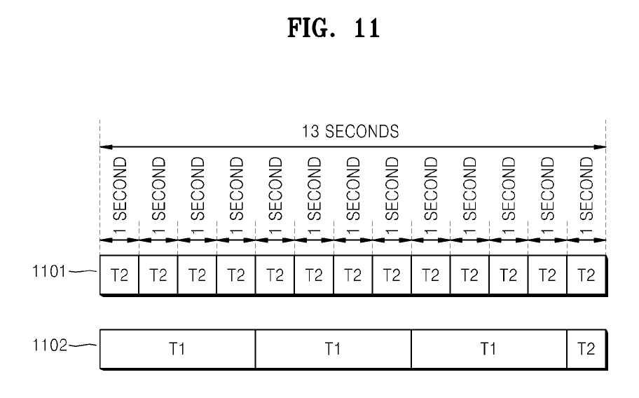

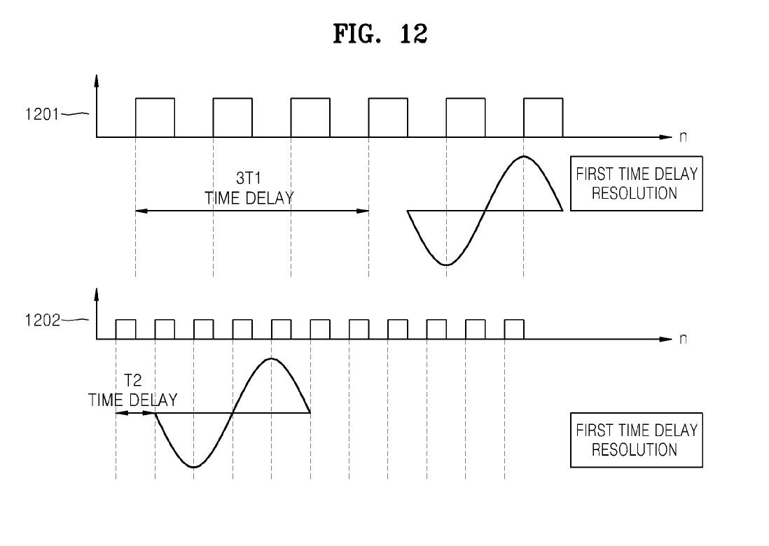

When the delay controller 707 receives the coordinate information relating to a focal point from the controller 706, the delay controller 707 calculates a respective time delay based on a corresponding distance between the focal point and each respective channel, and outputs information relating to a respective time delay to the received signal delayer 703. According to an exemplary embodiment, the clock generator 708 may generate a plurality of clock signals having different respective frequencies, and may provide the clock signals to the received signal delayer 703. The present exemplary embodiment will be described in detail with reference to FIG. 11. Hereinafter, functions of components will be described with reference to a process for determining a single focal point and performing scanning on the focal point which is executed via control performed by the controller 706.

The AD converter 709 converts an analog signal generated by the analog adder 704 into a digital signal, and then outputs the digital signal to the digital beamformer 710.

When the 1D transducer array 701 is an element of the 2D transducer array 202, a plurality of 1D transducer arrays 701 may be used. The digital beamformer 710 performs beamforming on a plurality of digital signals which have been processed by the AD converter 709, each of which corresponds to a respective one of the 1D transducer arrays 701. The digital beamformer 710 outputs the digital signal on which beamforming is performed to the image generating apparatus 403.

The residual signal remover 711 receives the coordinate information relating to the focal point from the controller 706 and removes a residual remaining signal while retaining an ultrasonic signal in the CCD which is included in the received signal delayer 703. In particular, a signal that is converted into image information is filtered by removing a signal that is received via each transducer, while preserving an ultrasonic signal.

The image generating apparatus 403 may receive the coordinate information relating to the focal point from the controller 706, may receive an intensity of a digital signal which corresponds to the focal point from the digital beamformer 710, and may temporally store the intensity of the digital signal as image information in a memory. When the image information relating to the focal point of an entire image is stored, the image generating apparatus 403 may output the image information to the image displaying apparatus 404.

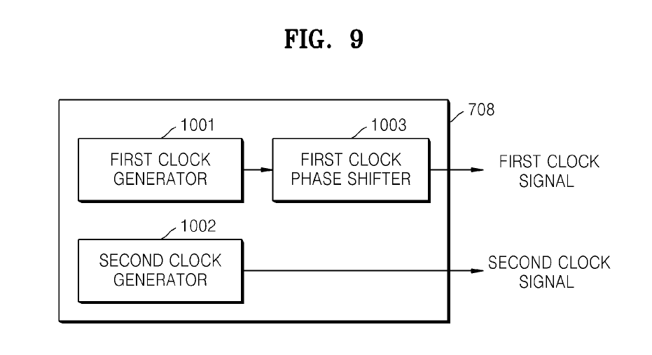

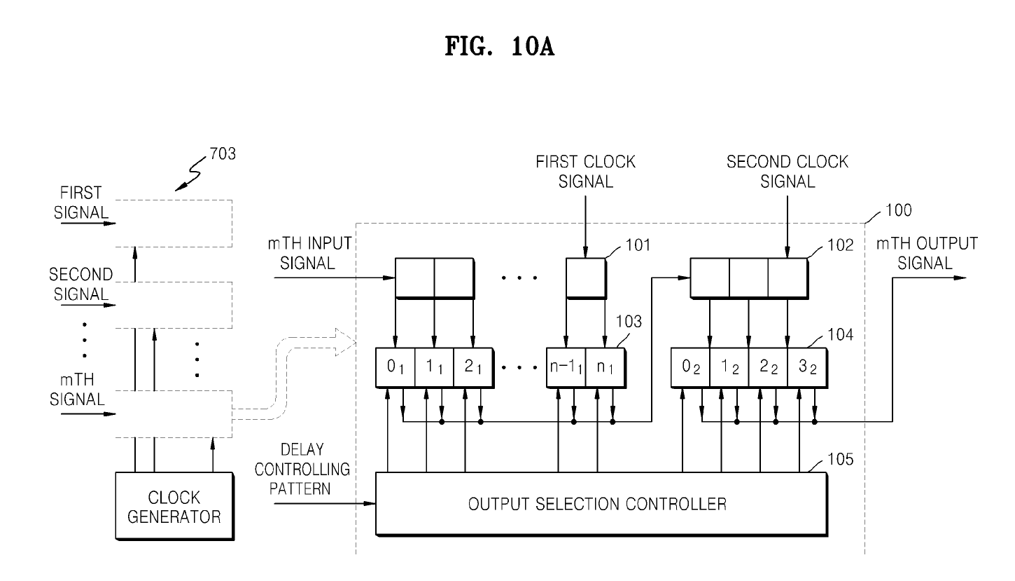

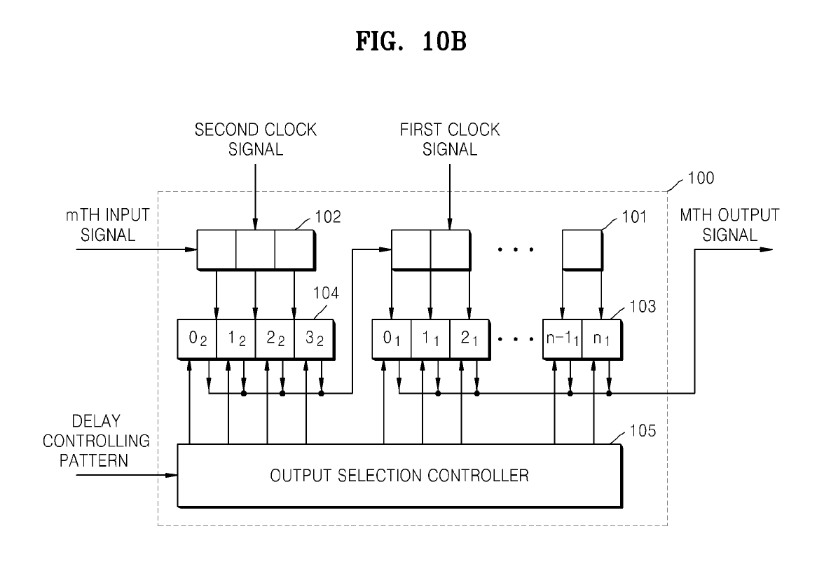

FIG. 9 is a structural diagram which illustrates the clock generator 708, according to an exemplary embodiment. The clock generator 708 of FIG. 5 includes a first clock generator 1001, a second clock generator 1002, and a first clock phase shifter 1003. The first clock generator 1001 provides a first clock signal to the first clock phase shifter 1003. The first clock phase shifter 1003 shifts a phase of the first clock signal which is received from the first clock generator 1001 by 0 degrees, 90 degrees, and 270 degrees. The first clock phase shifter 1003 may provide the shifted first clock signal to a first CCD delay device 101 (see FIGS. 10A and 10B) which is included in the received signal delayer 703. The second clock generator 1002 provides a second clock signal to a second CCD delay device 102 (see FIGS. 10A and 10B) which is also included in the received signal delayer 703.

FIGS. 10A and 10B are diagrams which illustrate a single channel 100 included in the received signal delayer 703 for correcting a time delay error, according to an exemplary embodiment. According to the present exemplary embodiment, the channel 100 of the received signal delayer 703 shown in FIG. 5 includes a first CCD delay device 101, a second CCD delay device 102, a first output selector 103, a second output selector 104, and an output selection controller 105. FIG. 10A illustrates a case in which an input signal is transmitted via the first CCD delay device 101 and the second CCD delay device 102 in the order stated. FIG. 10B illustrates a case in which an input signal is transmitted via the second CCD delay device 102 and the first CCD delay device 101 in the order stated.

The first CCD delay device 101 generates a plurality of outputs that are obtained by delaying input signals of respective channels based on the first clock signal by using the method described above with reference to FIG. 7. In this case, because the first clock signal has a lower time delay resolution than the second clock signal, when an input signal is delayed based on the first clock signal, a relatively large time delay effect is obtained by using the minimum time iteration (MTI) that is relatively large. The first output selector 103 receives a plurality of delayed signals from the first CCD delay device 101, and provides only one output from among the plurality of outputs, by using a selection which is based on the information received from the output selection controller 105, to the second CCD delay device 102. The second CCD delay device 102 generates a plurality of outputs that are obtained by delaying signals received from the first output selector 103 based on the second clock signal, and provides the plurality of outputs to the second output selector 104. Because the second clock signal has a higher time delay resolution than the first clock signal, a minimum time iteration for the second clock is smaller than a minimum time iteration for the first clock. Therefore, a resulting time delay effect is lesser with the second clock than with the first clock. In this case, because the second CCD delay device 102 has a relatively small time delay, a high time delay resolution may be obtained. According to the present exemplary embodiment, an operation of delaying a point of time by using a minimum time iteration that is relatively large, such as that used by the first clock signal, is referred to as “coarse time delay,” and an operation of delaying a point of time by using a minimum time iteration that is relatively small, such as that used by the second clock signal, is referred to as “fine time delay”. The second output selector 104 selects one signal from among a plurality of outputs received from the second CCD delay device 102, based on a signal received from the output selection controller 105. The output selection controller 105 may select one signal which is delayed by a desired degree, from among respective signals having different amounts of delay. The output selection controller 105 receives a delay controlling pattern from the delay controller 707, and outputs a switch operating signal which corresponds to a time delay which relates to the received delay controlling pattern to the first output selector 103 and the second output selector 104.

In this case, the number of clock signals is the same as the number of time delay resolutions used for a time delay. This is because time delay resolutions of each of the first and second CCD delay devices 101 and 102 are determined by clock signals. In FIGS. 10A and 10B, if a time delay resolution of the first CCD delay device 101 is f1 (as expressed in Hz), a time delay resolution f2 (as expressed in Hz) of the second CCD delay device 102 is determined to be equal to four times the value of f1. A first minimum time iteration (MTI) that is the MTI of the first CCD delay device 101 is T1 seconds, that is, the MTI of the “coarse time delay”. A second MTI that is the MTI of the second CCD delay device 102 is T2 seconds, that is, the MTI of the “fine time delay”. Thus, in this case, T1 is four times as great as T2.

The first output selector 103 and the second output selector 104 select signals of the “coarse time delay” and the “fine time delay”, respectively. Numbers indicated in the first output selector 103 and the second output selector 104 denote numbers of inputs of the first output selector 103 and the second output selector 104. A subscript denotes which output selector from among the first output selector 103 and the second output selector 104 is associated with the number. In particular, identification numbers 0₁, 1₁, 2₁, 3₁, through n₁, which use a common subscript 1, are associated with the first output selector 103. Identification numbers 0₂, 1₂, 2₂, and 3₂, which use a common subscript 2, are associated with the second output selector 104. The numbers of inputs of the first output selector 103 and the second output selector 104 are the same as the numbers of outputs of the first and second CCD delay devices 101 and 102, respectively. The numbers of the outputs of the first and second CCD delay devices 101 and 102 are related to a first time delay resolution and a second time delay resolution, respectively. The reasons for this will now be described. When a plurality of time delay resolutions are used, the number of CCD delay devices may be reduced. In particular, the number of CCD devices may be reduced by performing a single time delay which is equal to T1 instead of repeating a time delay which is equal to T2 four times, because T1 is four times as great as T2. Thus, when T1 is four times as great as T2, it is not required that the number of CCD delay devices which have an MTI of T2 exceeds 3. If the number of CCD delay devices is 3, four time delays of an output are 0 seconds, T0 seconds, 2T0 seconds, and 3T0 seconds, as illustrated in FIG. 7. Thus, when T1 is four times as great as T2, the number of inputs of the second output selector 104 may be equal to 4. Conversely, the number of the first CCD delay devices 101 may be varied based on a required total delay time. For example, the number of CCD delay devices may be determined in accordance with the number of time delays that must be performed by using the first MTI when a total time delay of 100 seconds is performed. Thus, when the number of the first CCD delay devices 101 is n and the number of the second CCD delay devices 102 is 3, a maximum time for performing a time delay is obtained according to Equation 1 below. For example, when T1 is 4 seconds and T2 is 1 second, if a required total time delay is 98 seconds, the number n of the first CCD delay devices 101 is 24 and the number of the second CCD delay devices 102 is 2.

(n×T₁)+T₂=n×(4T₂)+T₂=(4n+1)T₂ (1)

FIG. 11 is diagram which illustrates a method for correcting a time delay error by using the received signal delayer 703 as shown in FIGS. 10A and 10B, according to an exemplary embodiment. Reference number 1101 indicates the number of CCD delay devices that are required when a CCD delay device performs a time delay by using a conventional method. Reference number 1102 indicates the number of CCD delay devices that are required when a CCD delay device performs a time delay, according to an exemplary embodiment. In item 1102, the received signal delayer 703 performs a time delay by using a plurality of MTIs. In FIG. 11, the received signal delayer 703 performs a total time delay of 13 seconds. When the first time delay resolution is ¼ Hz, the first MTI is equal to T1 and T1 is 4 seconds. When the second time delay resolution is 1 Hz, the second MTI is equal to T2 and T2 is 1 second. As shown in item 1101, when the received signal delayer 703 uses the second time delay resolution, a total time delay effect of 13 seconds may be obtained by accumulating the delays provided by the second MTI for 13 times. When the received signal delayer 703 uses the first time delay resolution (T1=4 seconds), a total time delay effect of 12 seconds may be obtained by accumulating the delays provided by the first MTI for three times. However, compared with a time delay of 13 seconds, an error of 1 second may arise. Thus, the received signal delayer 703 may correct a time delay by using the second MTI together with the first MTI in order to correct the error. The received signal delayer 703 delays a signal by as much as an integral multiple of the first minimum time delay (T1=4 seconds) by using the first CCD delay device 101, and delays the delayed signal by as much as an integral multiple of the second minimum time delay (T2=1 second) by using the second CCD delay device 102. In particular, the received signal delayer 703 performs a total time delay by accumulating a plurality of minimum time delays. When a total time delay is performed by accumulating a plurality of minimum time delays, the number of CCD delay devices required by the received signal delayer 703 is 13 in item 1101 and is 4 in item 1102. Thus, when the received signal delayer 703 delays a signal, the number of required devices may be reduced.

FIG. 12 are diagrams which illustrate a case in which the received signal delayer 703 shown in FIGS. 10A and 10B performs a time delay by executing two processes, according to an exemplary embodiment. Reference number 1201 indicates a diagram which illustrates a case in which the first CCD delay device 101 performs a time delay by using the first time delay resolution (T1=4 seconds). In this case, a clock signal shown in an upper portion of diagram 1201 corresponds to the first clock signal shown in FIG. 10A. Reference number 1202 indicates a diagram which illustrates a case in which the second CCD delay device 102 performs a time delay with respect to the same signal upon which the time delay is performed in the process illustrated in diagram 1201 by using the second time delay resolution. In this case, a clock signal shown in an upper portion of diagram 1202 corresponds to the second clock signal shown in FIG. 10A. When the received signal delayer 703 performs the process illustrated in diagram 1201 only, because the process corresponds to the process shown in FIG. 8, a time delay error arises. However, when the process illustrated in diagram 1201 is performed and then the process illustrated in diagram 1202 is performed, a time delay error may be reduced or removed.

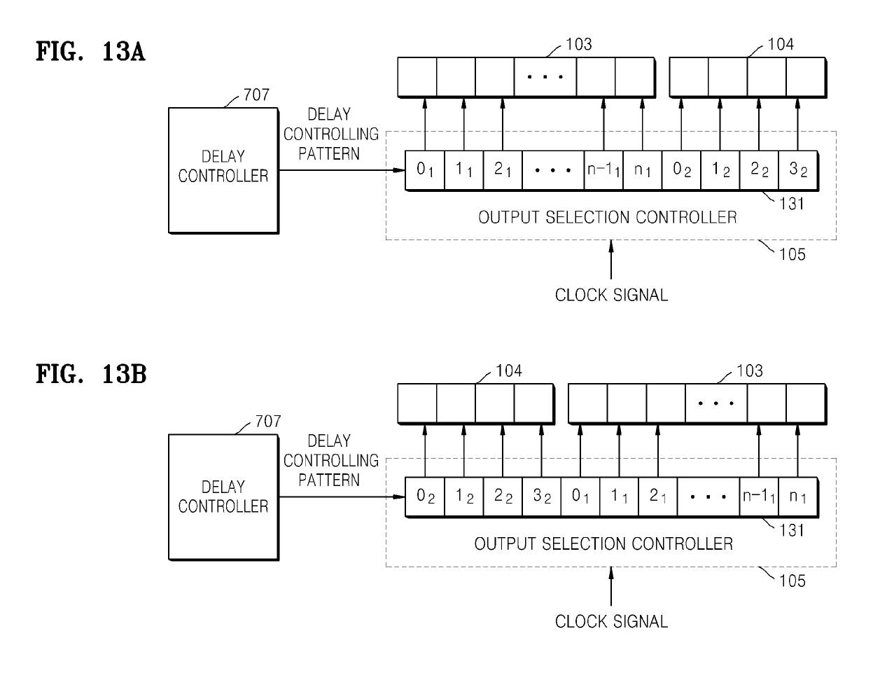

FIGS. 13A and 13B are diagrams which illustrate an internal structure of the output selection controller 105 shown in FIGS. 10A and 10B, according to exemplary embodiments. The output selection controller 105 includes a serial-in parallel-out (SIPO) shift register 131. When a respective plurality of signals are sequentially supplied to a single input terminal, the SIPO shift register 131 outputs respective signals from a corresponding plurality of outputs. A relationship between a delay control pattern and a delayed time of a practical signal will now be described. The first and second CCD delay devices 101 and 102 generate a plurality of outputs, each of which is delayed by an amount of time which is equal to an integral multiple of the corresponding MTI. In this case, the output selection controller 105 may select a signal that is delayed by a desired amount of time, from among the plurality of outputs. A process for selecting a signal by using the output selection controller 105 shown in FIGS. 13A and 13B will now be described in detail. The output selection controller 105 outputs selection signals to each of the first and second output selectors 103 and 104, respectively. The selection signals include signals for controlling the first and second output selectors 103 and 104, so as to cause a selection of respective inputs via each of the first and second output selectors 103 and 104. A selection signal for controlling one of the first or second output selector to select an output may be set to have a value which is equal to 1, and a selection signal for controlling one of the first or second output selector to discard an output may be set to have a value which is equal to 0. The output selection controller 105 serially and sequentially receives delay controlling patterns including 0 and 1 values from the delay controller 707, and transmits a signal for controlling each of the first output selector 103 and the second output selector 104 to a plurality of parallel outputs. For example, when the output selection controller 105 receives an input sequence of 0001 from the delay controller 707, four outputs which are respectively equal to 0, 0, 0, and 1 are transmitted to each of the first and second selectors 103 and 104. Each of the first and second selectors 103 and 104 receives selection signals from the output selection controller 105 and each selects and outputs one of a plurality of outputs of respective CCD delay devices, which selected outputs respectively correspond to each of the selection signals. In FIGS. 13A and 13B, numbers indicated in the output selection controller 105 indicate the number of switching signals output to the first and second selectors 103 and 104, and are the same as the numbers of outputs of the first and second CCD delay devices 101 and 102. A subscript is a number which refers to the particular selector which is indicated by the output selection controller 105. In particular, 0₁, 1₁, 2₁, through (n−1)₁, and n₁ are numbers which refer to the first output selector 103 that selects an output having a “coarse time delay”, and 0₂, 1₂, 2₂, and 3₂ are numbers which refer to the second output selector 104 that selects an output having a “fine time delay”. Because one signal is selected from each of the “fine time delay” and the “coarse time delay”, the output selection controller 105 may output the selection signal for selecting one signal from among 0₁ to n₁ and another signal from among 0₂ to 3₂. Thus, the delay controller 707 stores delay controlling patterns which respectively correspond to required delay times as data, as shown in Table 1 below. When the delay controller 707 receives coordinate information relating to a focal point from the controller 706, the delay controller 707 may calculate a delay time for each respective channel and may transmit a delay controlling pattern to the output selection controller 105.

TABLE 1

| | delay controlling pattern | | | | | | | | | |
|---|---|---|---|---|---|---|---|---|---|---|
| | first output | | | | | | | | | |
| | selector | | | | second output | | | | | |
| | (coarse time | | | | selector | | | | | relative |
| pattern | delay) | | | | (fine time delay) | | | | | delay |
| number | 0 | 1 | . . . | n | 0 | 1 | 2 | 3 | delayed time | size |
| 1 | 1 | 0 | . . . | 0 | 1 | 0 | 0 | 0 | 0 | small |
| 2 | 1 | 0 | . . . | 0 | 0 | 1 | 0 | 0 | T2 | |
| 3 | 1 | 0 | . . . | 0 | 0 | 0 | 1 | 0 | 2T2 | |
| 4 | 1 | 0 | . . . | 0 | 0 | 0 | 0 | 1 | 3T2 | |
| 5 | 0 | 1 | . . . | 0 | 1 | 0 | 0 | 0 | T1 | |
| 6 | 0 | 1 | . . . | 0 | 0 | 1 | 0 | 0 | T1 + T2 | |
| 7 | 0 | 1 | . . . | 0 | 0 | 0 | 1 | 0 | T1 + 2T2 | |
| 8 | 0 | 1 | . . . | 0 | 0 | 0 | 0 | 1 | T1 + 3T2 | |
| 9 | 0 | 0 | . . . | 0 | 1 | 0 | 0 | 0 | 2T1 | |
| 10 | 0 | 0 | . . . | 0 | 0 | 1 | 0 | 0 | 2T1 + T2 | |
| 11 | 0 | 0 | . . . | 0 | 0 | 0 | 1 | 0 | 2T1 + 2T2 | |
| 12 | 0 | 0 | . . . | 0 | 0 | 0 | 0 | 1 | 2T1 + 3T2 | |
| . | . | | | | . | | | | . | |
| . | . | | | | . | | | | . | |
| . | . | | | | . | | | | . | |
| 4n + 1 | 0 | 0 | . . . | 1 | 1 | 0 | 0 | 0 | nT1 | |
| 4n + 2 | 0 | 0 | . . . | 1 | 0 | 1 | 0 | 0 | nT1 + T2 | |
| 4n + 3 | 0 | 0 | . . . | 1 | 0 | 0 | 1 | 0 | nT1 + 2T2 | |
| 4n + 4 | 0 | 0 | . . . | 1 | 0 | 0 | 0 | 1 | nT1 + 3T2 | great |

The leftmost column of Table 1 indicates a sequential pattern order. A second column from the right side indicates a total delayed time. The delay controller 707 may compare a required total time delay with a result of the second column from the right side, and may use this comparison to determine a corresponding delay controlling pattern.

In a structure in which an input signal is first received the second CCD delay device 102, similarly as illustrated in FIG. 10B, the output selection controller 105 has a structure as shown in FIG. 13B. In this case, when a signal 1 is supplied to a selected switch device and a signal 0 is supplied to each of the remaining switch devices, the delay controller 707 may store a delay controlling pattern, as shown in Table 2 below.

TABLE 2

| | delay controlling pattern | | | | | | | | | |
|---|---|---|---|---|---|---|---|---|---|---|
| | second output | | | | first output | | | | | |
| | selector | | | | selector (coarse | | | | | relative |
| pattern | (fine time delay) | | | | time delay) | | | | | delay |
| number | 0 | 1 | 2 | 3 | 0 | 1 | . . . | N | delayed time | size |
| 1 | 1 | 0 | 0 | 0 | 1 | 0 | . . . | 0 | 0 | small |
| 2 | 0 | 1 | 0 | 0 | 1 | 0 | . . . | 0 | T2 | |
| 3 | 0 | 0 | 1 | 0 | 1 | 0 | . . . | 0 | 2T2 | |
| 4 | 0 | 0 | 0 | 1 | 1 | 0 | . . . | 0 | 3T2 | |
| 5 | 1 | 0 | 0 | 0 | 0 | 1 | . . . | 0 | T1 | |
| 6 | 0 | 1 | 0 | 0 | 0 | 1 | . . . | 0 | T1 + T2 | |
| 7 | 0 | 0 | 1 | 0 | 0 | 1 | . . . | 0 | T1 + 2T2 | |
| 8 | 0 | 0 | 0 | 1 | 0 | 1 | . . . | 0 | T1 + 3T2 | |
| 9 | 1 | 0 | 0 | 0 | 0 | 0 | . . . | 0 | 2T1 | |
| 10 | 0 | 1 | 0 | 0 | 0 | 0 | . . . | 0 | 2T1 + T2 | |
| 11 | 0 | 0 | 1 | 0 | 0 | 0 | . . . | 0 | 2T1 + 2T2 | |
| 12 | 0 | 0 | 0 | 1 | 0 | 0 | . . . | 0 | 2T1 + 3T2 | |
| . | . | | | | . | | | | | |
| . | . | | | | . | | | | | |
| . | . | | | | . | | | | | |
| 4n + 1 | 1 | 0 | 0 | 0 | 0 | 0 | . . . | 1 | nT1 | |
| 4n + 2 | 0 | 1 | 0 | 0 | 0 | 0 | . . . | 1 | nT1 + T2 | |
| 4n + 3 | 0 | 0 | 1 | 0 | 0 | 0 | . . . | 1 | nT1 + 2T2 | |
| 4n + 4 | 0 | 0 | 0 | 1 | 0 | 0 | . . . | 1 | nT1 + 3T2 | great |

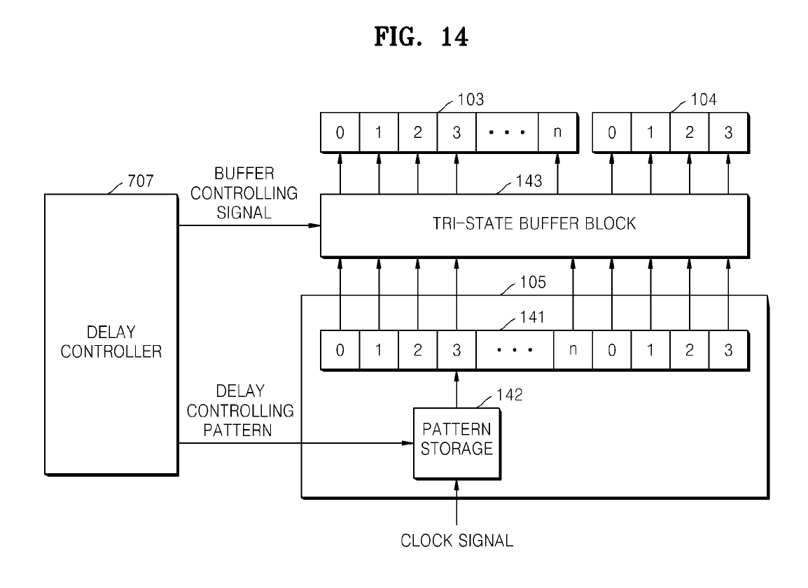

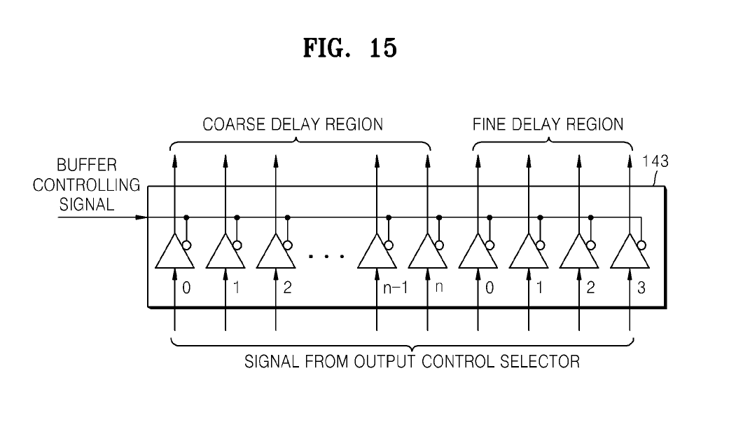

FIG. 14 is a diagram which illustrates an internal structure of the output selection controller 105 shown in FIGS. 10A and 10B, according to another exemplary embodiment. The output selection controller 105 includes a selection signal outputting unit 141 and a pattern storage 142. The delay controller 707 repeatedly performs a storing process and a scanning process. In the storing process, a delay controlling pattern relating to a plurality of focal points and a storing address are stored together in the pattern storage 142. A tri-state buffer block 143 prevents the output selection controller 105 and the first and second output selectors 103 and 104 from being connected to each other during the storing process, but does not prevent such a connection during the scanning process. A buffer controlling signal for blocking a buffer is received from the delay controller 707 in order to perform the prevention of connection and the non-prevention. A tri-state buffer is a type of logic device. The tri-state buffer includes a data input and a buffer controlling signal input as inputs, and a data output as an output. The tri-state buffer block 143 includes a tri-state buffer which has an internal structure which is illustrated in FIG. 15. A buffer controlling signal for blocking a buffer may have a value which is set to be equal to 0, and a buffer controlling signal for not blocking may have a value which is set to be equal to 1. When the tri-state buffer block 143 receives a buffer controlling signal for blocking the buffer from the delay controller 707, a connection between the output selection controller 105 and the first and second output selectors 103 and 104 is blocked. After the tri-state buffer block 143 blocks the buffer, when the delay controller 707 provides a plurality of delay controlling patterns and an address to the pattern storage 142, the pattern storage 142 sequentially stores delay controlling patterns which correspond to respective addresses. In this case, when delay controlling patterns are stored in an order which relates to a pattern number and/or a depth direction order of a focal point in a single scan line as illustrated in FIG. 4, as shown in Table 3 (when a coarse time delay is first performed), the delay controlling patterns may be sequentially accessed based on an address during scanning of an image, and thus, an image having a high frame rate may be obtained. After the storing process is terminated, the scanning process is begun. The scanning process includes a process in which delay controlling patterns are sequentially output and beamforming is performed on a plurality of focal points. In the scanning process, the delay controlling patterns which are stored in the pattern storage 142 are sequentially read in an order relating to an address, and the delay controlling patterns are transmitted to the selection signal outputting unit 141. The selection signal outputting unit 141 sequentially outputs this signal to each of the first and second output selectors 103 and 104.

TABLE 3

| | | | | | | | | | depth |
|---|---|---|---|---|---|---|---|---|---|
| | stored delay data | | | | | | | | direction |
| address | coarse time delay | | | | fine time delay | | | | of |
| number | 0 | 1 | . . . | n | 0 | 1 | 2 | 3 | focal point |
| #1 | 1 | 0 | . . . | 0 | 1 | 0 | 0 | 0 | deep |
| #2 | 1 | 0 | . . . | 0 | 0 | 1 | 0 | 0 | |
| #3 | 1 | 0 | . . . | 0 | 0 | 0 | 1 | 0 | |
| #4 | 1 | 0 | . . . | 0 | 0 | 0 | 0 | 1 | |
| #5 | 0 | 1 | . . . | 0 | 1 | 0 | 0 | 0 | |
| #6 | 0 | 1 | . . . | 0 | 0 | 1 | 0 | 0 | |
| . | . | . | . | . | . | . | . | . | |
| . | . | . | . | . | . | . | . | . | |
| . | . | . | . | . | . | . | . | . | |
| #(2^{k} − 2) | 0 | 0 | . . . | 1 | 0 | 1 | 0 | 0 | |
| #(2^{k} − 1) | 0 | 0 | . . . | 1 | 0 | 0 | 1 | 0 | |
| #(2^{k}) | 0 | 0 | . . . | 1 | 0 | 0 | 0 | 1 | shallow |

When the fine time delay is first performed, delay data stored in the pattern storage 142 may be stored in a form as shown in Table 4 below.

TABLE 4

| | | | | | | | | | depth |
|---|---|---|---|---|---|---|---|---|---|
| | stored delay data | | | | | | | | direction |
| address | coarse time delay | | | | coarse time delay | | | | of |
| number | 0 | 1 | 2 | 3 | 0 | 1 | . . . | n | focal point |
| #1 | 1 | 0 | 0 | 0 | 1 | 0 | . . . | 0 | deep |
| #2 | 0 | 1 | 0 | 0 | 1 | 0 | . . . | 0 | |
| #3 | 0 | 0 | 1 | 0 | 1 | 0 | . . . | 0 | |
| #4 | 0 | 0 | 0 | 1 | 1 | 0 | . . . | 0 | |
| #5 | 1 | 0 | 0 | 0 | 0 | 1 | . . . | 0 | |
| #6 | 0 | 1 | 0 | 0 | 0 | 1 | . . . | 0 | |
| . | . | . | . | . | . | . | . | . | |
| . | . | . | . | . | . | . | . | . | |
| . | . | . | . | . | . | . | . | . | |
| #(2^{k}−)2 | 0 | 1 | 0 | 0 | 0 | 0 | . . . | 1 | |
| #(2^{k} − 1) | 0 | 0 | 1 | 0 | 0 | 0 | . . . | 1 | |
| #(2^{k}) | 0 | 0 | 0 | 1 | 0 | 0 | . . . | 1 | shallow |

FIG. 15 is a diagram which illustrates an internal structure of the tri-state buffer block 143 shown in FIG. 14, according to an exemplary embodiment. When a plurality of delay patterns are received from the output selection controller 105, the received delay patterns may be transmitted or blocked based on a buffer controlling signal. In particular, the tri-state buffer block 143 of FIG. 14 maintains a state of preventing the first and second output selectors 103 and 104 and the output selection controller 105 from being connected to each other, during the storing process. A plurality of inputs and a plurality of outputs of the tri-state buffer block 143 are connected by tri-state buffer devices. A buffer controlling signal opens and closes a data input. When the buffer controlling signal is equal to 1, the three-state buffer simply amplifies the received data input with an amplification ratio of 1 and outputs the amplified data input to an output. When the buffer controlling signal is equal to 0, the three-state buffer does not output any output, even if the data input has been received. The delay controller 707 shown in FIG. 14 may supply a buffer controlling signal having a value of “0” to the tri-state buffer block 143 during the storing process of storing data in the pattern storage 142, and may thusly prevent the first and second output selectors 103 and 104 and the output selection controller 105 from being connected to each other. After the storing process has been completed, the delay controller 707 supplies a buffer controlling signal having a value of “1” to the tri-state buffer block 143, and may thusly enable a connection between the first and second output selectors 103 and 104 and the output selection controller 105 to each other, during the scanning process illustrated in FIG. 14.

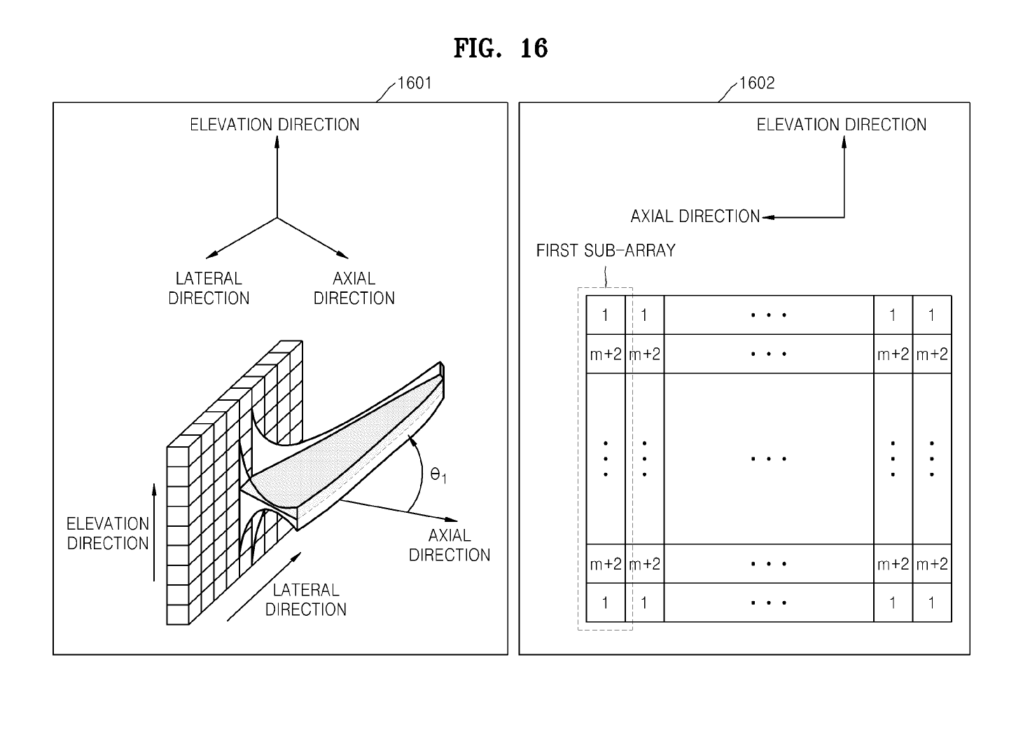

FIG. 16 is a diagram which illustrates a method for obtaining a planar image by using a 2D transducer array, according to an exemplary embodiment. If a lateral direction is indicated by an x axis, an elevation direction is indicated by a y axis, and an axial direction is indicated by a z axis, an angle between an x-z plane and a target plane may be indicated by θ. In order to obtain an image on a predetermined plane having an angle which is equal to θ1, a time delay must be performed on all transducer elements of the 2D transducer array. Reference number 1602 indicates a 2D transducer array which is illustrated in the drawing indicated by reference number 1601 based on an x-y plane. In item 1602, a 1D array of the elevation direction (as indicated by the y axis) is set to be a sub-array and numbers indicated in transducer elements are pattern numbers which are similar to those shown in Table 1 and Table 2. In addition, numbers may be determined from the leftmost column and may be set to be a first sub-array, a second sub-array, and so on, respectively. Because all sub-arrays have the same angle θ, all of the sub-arrays have the same time delay pattern. In item 1602, time delay patterns of sub-arrays are shown to be the same. Because a single image corresponds to a single angle θ, when N angles θ are set, N images may be obtained. In order to control a time delay pattern in a single sub-array, when control data of K bits is used, a corresponding image may be controlled based on 2^{K} permutations of bits. For example, assuming that K=3, the delay controller 707 may store a time delay pattern of a sub-array corresponding to data as shown in Table 5 below.

TABLE 5

| | 3 bit number | | | time delay pattern of sub-array | | | | | | |
|---|---|---|---|---|---|---|---|---|---|---|
| θ | #1 | #2 | #3 | E₁ | E₂ | E₃ | . . . | E_{N} | E_{N-1} | E_{N} |
| θ1 | 0 | 0 | 0 | 1 | 4 | 10 | . . . | 10 | 4 | 1 |
| θ2 | 0 | 0 | 1 | 1 | 4 | 9 | . . . | 9 | 4 | 1 |
| θ3 | 0 | 1 | 0 | 1 | 4 | 8 | . . . | 8 | 4 | 1 |
| θ4 | 0 | 1 | 1 | 1 | 3 | 8 | . . . | 8 | 3 | 1 |
| θ5 | 1 | 0 | 0 | 1 | 3 | 6 | . . . | 7 | 3 | 1 |
| θ6 | 1 | 0 | 1 | 1 | 3 | 5 | . . . | 5 | 3 | 1 |
| θ7 | 1 | 1 | 0 | 1 | 2 | 5 | . . . | 5 | 2 | 1 |
| θ8 | 1 | 1 | 1 | 1 | 2 | 4 | . . . | 4 | 2 | 1 |

In Table 5 above, E₁ to E_{N} are respective transducer elements of a sub-array and N is the number of the transducer elements in the sub-array. Because a number of cases which is equal to 2³ may be obtained by using 3 bits, time delay patterns of a sub-array of 8 planes may correspond to the number of cases of 2³, and thus, the delay controller 707 may control 2^{K} planes when K bits are used.

The exemplary embodiments can be written as computer programs and can be implemented in general-use digital computers that execute the programs by using a transitory or a non-transitory computer readable recording medium. Examples of the computer readable recording medium include magnetic storage media (e.g., read-only memory (ROM), floppy disks, hard disks, etc.), and storage media such as optical recording media (e.g., compact disk-read-only memory (CD-ROMs), or digital versatile disks (DVDs)).

As described above, according to the one or more of the above exemplary embodiments, in an analog beamforming method, beamforming may be performed by using a small number of circuit devices, the size of a system may be reduced and the amount of calculation may be reduced by comparison with conventional beamforming systems. In addition, during the beamforming, the number of switching operations may be reduced to minimize noise.

It should be understood that the exemplary embodiments described herein should be considered in a descriptive sense only and not for purposes of limitation. Descriptions of features or aspects within each exemplary embodiment should typically be considered as available for other similar features or aspects in other exemplary embodiments.

What is claimed is:

1. An apparatus for performing analog beamforming by using ultrasonic signals which are received by a transducer array having a plurality of transducer elements, the apparatus comprising:
a delay controller which outputs respective first delay controlling information which indicates a number of delays of a first time interval and respective second delay controlling information which indicates a number of delays of a second time interval, with respect to each respective one of the ultrasonic signals and a respective predetermined position of a scanning region;
first analog devices, wherein each respective one of the first analog devices selectively delays a corresponding one of the received ultrasonic signals by a respective first amount of time which is less than or equal to the first time interval at least once, based on the corresponding first delay controlling information;
second analog devices, wherein each respective one of the second analog devices selectively delays a corresponding one of the selectively delayed ultrasonic signals by a respective second amount of time which is less than or equal to the second time interval at least once, based on the corresponding second delay controlling information; and
an adder which sums the ultrasonic signals which have been selectively delayed by the second analog devices.

2. The apparatus of claim 1, wherein the first time interval is greater than the second time interval.

3. The apparatus of claim 1, wherein the second time interval is greater than the first time interval.

4. The apparatus of claim 1, further comprising a clock generator which generates a plurality of clock signals having different frequencies,
wherein each respective one of the first analog devices selectively delays the corresponding one of the received ultrasonic signals at least once, based on a first clock signal from among the plurality of clock signals and the corresponding first delay controlling information, and
wherein each respective one of the second analog devices selectively delays the corresponding one of the ultrasonic signals which have been selectively delayed by the first analog devices at least once, based on a second clock signal from among the plurality of clock signals and the corresponding second delay controlling information.

5. The apparatus of claim 4, wherein the clock generator comprises:
a basic clock generator which generates a plurality of basic clock signals having different frequencies; and
a clock phase shifter which shifts a phase of at least one of the plurality of basic clock signals,
wherein the plurality of clock signals includes each of the plurality of basic clock signals and each of the at least one of the plurality of basic clock signals having a shifted phase.

6. The apparatus of claim 1, further comprising:
a controller which determines respective time points of time when each respective one of the ultrasonic signals is received by a corresponding one of the plurality of transducer elements; and
a switch which switches a respective connection between each respective one of the plurality of transducer elements and a corresponding one of the first analog devices, based on the determined respective time points.

7. The apparatus of claim 1, wherein the delay controller comprises:
a delay time outputting unit which determines respective delay times which are applied to each respective one of the ultrasonic signals such that each respective one of the ultrasonic signals reaches the corresponding predetermined position of the scanning region simultaneously; and
a control information outputting unit which outputs the respective first delay controlling information and the respective second delay controlling information, which correspond to the determined respective delay times.

8. The apparatus of claim 7, wherein the control information outputting unit comprises:
a storage which associates each respective one of the determined delay times to the corresponding first delay controlling information and the corresponding second delay controlling information, and which stores each respective one of the determined delay times together with the corresponding first delay controlling information and the corresponding second delay controlling information; and
a controlling information searcher which outputs the respective first delay controlling information and the respective second delay controlling information from among the first delay controlling information and the second delay controlling information which are stored in the storage.

9. The apparatus of claim 8, wherein the storage associates each respective one of the determined delay times to the corresponding first delay controlling information and the corresponding second delay controlling information and stores each respective one of the determined delay times together with the corresponding first delay controlling information and the corresponding second delay controlling information based on an order of the determined delay times.

10. An ultrasonic scanning system comprising:
two-dimensional (2D) transducer array having two or more one-dimensional (1D) transducer arrays in which a plurality of transducer elements are arranged in one of a horizontal direction and a vertical direction; and
a three-dimensional (3D) beamforming performing apparatus which performs analog beamforming in a first direction which is selected from the horizontal direction and the vertical direction by using the apparatus of claim 1, and which performs digital beamforming in a second direction which is different from the first direction.

11. A method for performing analog beamforming by using ultrasonic signals that are received by a transducer array having a plurality of transducer elements, the method comprising:
obtaining respective first delay controlling information which indicates a number of delays of a first time interval and respective second delay controlling information which indicates a number of delays of a second time interval, with respect to each respective one of the ultrasonic signals and a respective predetermined position of a scanning region;
selectively delaying each respective one of the received ultrasonic signals by a respective first amount of time which is less than or equal to the first time interval at least once, based on the corresponding first delay controlling information;
selectively delaying each respective one of the delayed ultrasonic signals by a respective second amount of time which is less than or equal to the second time interval at least once, based on the corresponding second delay controlling information; and
summing the ultrasonic signals which have twice been selectively delayed.

12. The method of claim 11, wherein the first time interval is greater than the second time interval.

13. The method of claim 11, wherein the second time interval is greater than the first time interval.

14. The method of claim 11, further comprising generating a first clock signal which has a first frequency and a second clock signal which has a second frequency which is different from the first frequency,
wherein the delaying the received ultrasonic signals comprises: selectively delaying each respective one of the received ultrasonic signals at least once, based on the corresponding first delay controlling information and the first clock signal, and
wherein the delaying the delayed ultrasonic signals comprises: selectively delaying each respective one of the delayed ultrasonic signals at least once, based on the corresponding second delay controlling information and the second clock signal.

15. The method of claim 14, wherein the generating the first clock signal and the second clock signal comprises
shifting a phase of at least one of the first clock signal and the second clock signal.

16. The method of claim 11, further comprising associating each respective predetermined position of the scanning region to the corresponding first delay controlling information and the corresponding second delay controlling information and storing each respective predetermined position of the scanning region together with the associated corresponding first delay controlling information and the associated corresponding second delay controlling information,
wherein the obtaining the respective second delay controlling information comprises outputting the respective first delay controlling information and the respective second delay controlling information based on the corresponding associated predetermined position of the scanning region.

17. The method of claim 11, wherein the transducer elements are arranged in a horizontal direction and a vertical direction; and
wherein the analog beamforming is performed in a first direction which is selected from the horizontal direction and the vertical direction and digital beamforming is performed in a second direction which is different from the first direction.

18. A non-transitory computer readable recording medium having recorded thereon a program for executing the method of claim 11.

19. The apparatus of claim 1, wherein each of the first analog devices and each of the second analog devices is a charge-coupled device.

20. The apparatus of claim 19, further comprising a residual signal remover which removes a respective signal that exits from each respective one of the first analog devices and from each respective one of the second analog devices after each corresponding one of the received ultrasonic signals is selectively delayed.
